(19) United States
(12) Patent Application Publication
(10) Pub. No.: US 2015/0132790 A1
(43) Pub. Date: May 14, 2015

(54) SCREEN TO IDENTIFY AGENTS THAT CAN MODULATE HEME TRANSPORTER

(71) Applicants: University of Maryland, College Park, College Park, MD (US); The University of Texas Southwestern Medical Center, Dallas, TX (US)

(72) Inventors: Iqbal Hamza, Kensington, MD (US); Richard Bruick, Dallas, TX (US)

(21) Appl. No.: 14/572,885

(22) Filed: Dec. 17, 2014

Related U.S. Application Data
(63) Continuation of application No. 13/864,545, filed on Apr. 17, 2013.
Application No. 13/864,545 is a continuation-in-part of application No. 12/068,664, filed on Feb. 8, 2008, now abandoned.
Application No. 12/068,664 is a continuation of application No. 11/259,248, filed on Oct. 27, 2005, now abandoned.
(60) Provisional application No. 61/782,082, filed on Mar. 14, 2013.
(60) Provisional application No. 61/625,217, filed on Apr. 17, 2012.
(60) Provisional application No. 60/652,975, filed on Feb. 15, 2005.

(51) Int. Cl. G01N 33/50

(52) U.S. Cl. CPC G01N 33/5032
USPC 435/29

(57) ABSTRACT

Provided are compositions and methods related to heme transporters and high-throughput methods of identifying agents that can modulate heme transporters. An approach for identifying a modulator of a eukaryotic heme transporter involves adding a toxic heme analog and at least one test agent to a culture of cells, wherein the cells express a recombinant heterologous eukaryotic heme transporter. The cells are incubated with the toxic heme analog and the test agent for a period of time. A change in toxic effect of the toxic heme analog relative to a control is indicative that the test agent is a modulator of the eukaryotic heme transporter. Heme transporter agonists and antagonists can be identified. Also provided is a cell culture comprising a plurality of cells which express a recombinant heterologous eukaryotic heme transporter. The plurality of cells are divided into a plurality of reaction chambers, each of which may contain a test agent, and may further contain a toxic heme analog.

### CROSS REFERENCE TO RELATED APPLICATIONS

The present application is a continuation of U.S. patent application Ser. No. 13/864,545, filed Apr. 17, 2013, which claims priority to U.S. provisional patent application no. 61/625,217, filed Apr. 17, 2012, and to U.S. provisional patent application no. 61/782,082, filed on Mar. 14, 2013. The present application is also a continuation-in-part of U.S. patent application Ser. No. 12/068,664, filed Feb. 8, 2008, which is a continuation of U.S. patent application Ser. No. 11/259,248, filed Oct. 27, 2005, which claims priority to U.S. provisional patent application no. 60/652,975, filed Feb. 15, 2005. The disclosures of each of the afore-mentioned applications are incorporated herein by reference.

### FIELD

The present invention relates generally to compositions and methods for combating parasitic infections, and more specifically to identifying agents that can inhibit heme transporter(s) that have relevance for parasitic infections in humans and non-human animals.

### BACKGROUND

Iron deficiency is the most common nutritional disorder, affecting as many as four out of five people worldwide. Even though iron is one of the most abundant elements in the earth's crust it is not readily bioavailable for absorption in the human intestine. Heme (iron-protoporphyrin IX) is a significant source of bioavailable iron for enterocytes where heme-iron is absorbed and for macrophages where heme-iron is recycled. In the human intestine, dietary heme is more easily absorbed than inorganic iron and is the source for two-thirds of body iron in meat-eating individuals. Moreover, >60% of the total body iron is present as heme in hemoglobin. Iron from heme is recycled by phagocytosis of senescent red blood cells in the phagolysosome of macrophages. As of yet, the genes and pathways responsible for heme transport in human enterocytes and macrophages remain unknown. Since heme is a hydrophobic and cytotoxic macrocycle, it likely does not passively diffuse through membranes but is instead actively transported via specific intra- and inter-cellular pathways.

In an effort to ultimately define and characterize the cellular and molecular determinants of heme homeostasis in human health and disease, studies have been initiated in the roundworm, Caenorhabditis elegans. Worms are an excellent genetic animal model to identify components of the heme transport pathways because they do not synthesize heme. Nevertheless, C. elegans synthesizes a large number of hemoproteins with human homologs and therefore requires dietary heme for growth and reproduction. Thus, the worm model provides a clean genetic background devoid of endogenous heme and the ability to externally manipulate the metabolic flux of intracellular heme. This approach recently resulted in the discovery of HRG-1 and its paralog HRG-4, the first eukaryotic heme importers/transporters. HRG-1 is a permease that is functionally conserved in humans and binds and transports heme. The worm and human HRG-1 proteins co-localize to the endo-lysosomal compartment, while their paralog HRG-4 localizes to the plasma membrane. These studies established a conserved model for cellular heme transport and validated C. elegans as a bona fide prototype for the characterization of heme homeostasis pathways. However, there are no previously available pharmacological tools to aid in the study of the cellular and physiological roles of these eukaryotic heme transporters in a convenient, high throughput manner. The present invention meets this and other needs.

### SUMMARY

The present disclosure provides compositions and methods for identification of agents that can modulate the function of eukaryotic heme transporters. In particular embodiments, the method facilitates identification of agonists or antagonists of heme transporters. The disclosure includes approaches which involve making and/or using single-celled or multi-cellular eukaryotic organisms as a system to express and test the function of heme importers or exporters that are not endogenously expressed by the cells. The cells are thus engineered to recombinantly express a heterologous heme transporter, which is referred to herein in certain embodiments as a heme responsive gene (“HRG”) transporter. The cells can be prokaryotic or eukaryotic. In one embodiment, the system involves single-celled eukaryotic organisms. In one embodiment, the single celled organisms are yeast. The disclosure includes introducing expression vector(s) into one or more cells, wherein the expression vector(s) express an HRG transporter.

In embodiments the present disclosure includes a method of identifying a modulator of a eukaryotic heme transporter. The method may comprise adding a toxic heme analog and at least one test agent to a culture of cells, wherein the cells express a recombinant heterologous eukaryotic heme transporter. The toxic heme analog and the test agent may be added concurrently or consecutively. In embodiments, the toxic heme analog is added before the test agent. In embodiments, the toxic heme analog is added after the test agent. The culture of cells can be incubated for a period of time before adding the test agent, and the cell culture and the toxic heme analog and the test agent may be incubated together for a period of time. The incubations can be performed for any desirable amount of time, such as from at least one minute, to at least 1-16 hours, including all time values there between to the minute, and all ranges there between, or over a period of at least one to several days. The incubation can be performed at any desirable temperature, with any other desirable conditions, such as controlled humidity, air flow and the like. Observing a change in toxic effect of the toxic heme analog relative to a control is indicative that the test agent is a modulator of the eukaryotic heme transporter. In embodiments, the toxic heme analog comprises gallium.

The cell culture can be a liquid or solid medium or semi-solid medium, such as a liquid cell culture, or semi-solid culture medium of the type used in a petri or other culture dish. In embodiments, the cell cultures comprises a liquid culture which is separated into a plurality of reaction chambers, such as in a high-throughput configuration. In an embodiment, the plurality of reaction chambers comprises up to or at least 384 reaction chambers. Into each reaction chamber the toxic heme analog and a distinct test agent may be added, and a change in the cell culture due to the presence of the test agent can be observed. In an embodiment, a change in the toxic effect of the toxic heme analog comprises reduced lethality of the toxic heme analog, which thereby identifies the test agent as an antagonist of the eukaryotic heme transporter.

In embodiments, the recombinant heterologous eukaryotic heme transporter is a eukaryotic heme transporter that is endogenously expressed by a parasite of humans and/or or non-human animals.

The disclosure also provides a cell culture comprising a plurality of cells which express a recombinant heterologous eukaryotic heme transporter, wherein the plurality of cells are divided into a plurality of reaction chambers, which may further comprise a test agent and/or a toxic heme analog.

### BRIEF DESCRIPTION OF THE FIGURES

FIG. 1. Photographic representation of HRG-4 transport the toxic heme analog GaPPIX in wild-type yeast. Transformed wild-type w303 yeast were serially diluted and plated on raffinose/galactose minimal SD -Ura agar plates supplemented with either no or 1 μM GaPPIX to determine growth after 3 and 5 days.

FIG. 2. Graphical depiction of dose response for GaPPIX toxicity in wild-type yeast expressing HRG-4. Transformed wild-type w303 yeast cells were inoculated at 0.1 OD600 in 10 mL of SD, minus Ura, plus raffinose and galactose medium and the indicated concentrations of GaPPIX for 16 h prior to determining growth. The relative IC50 (mean±SEM) for Vector was 61.77 μM and CeHRG-4 was 0.029 μM.

### DETAILED DESCRIPTION

The present disclosure provides compositions and methods that are useful for identification of agents that can affect function of eukaryotic heme transporters. In particular embodiments described herein, the method is suitable for identification of modulators (i.e., antagonists and/or agonists) of the heme responsive gene tranporters known in the art as “HRG”s. See, for example, Rajagopal A et al, Nature 2008, 453(7198):1127-1131; and US Patent Publication 20090093377, from which the description of HRGs is incorporated herein by reference. Any heme transporter that is endogenously expressed by a human or non-human animal parasite can be expressed heterologously via compositions and methods provided by the present disclosure.

Test agents identified using compositions and methods of this disclosure will have relevance to human and non-human animals. In certain embodiments, compounds identified using the compositions and methods of will be useful for, among other purposes, inhibiting reproduction and/or growth of certain parasites, and for prophylaxis and/or therapy for disorders that relate to iron deficiencies or to iron overload. Thus, the present disclosure relates to in embodiments a system to identify agents which will be used in pharmaceutical approaches directed to a variety of conditions relating to abnormal heme metabolism.

In general, this disclosure provides approaches which include making and using single-celled or multi-cellular eukaryotic organisms as a system to express and test the function of heme transporters (heme importers or exporters) that are not normally endogenously expressed by the cells, and are thus heterologous relative to the cells that express them recombinantly. In embodiments, a heme transporter that is heterologous to a cell that has been engineered to express it is a heme transporter that is not encoded by the genome of the cell. In one embodiment, the single-celled organisms are yeast, such as S. cerevisiae.

Any expression vector can be used and will be dependent upon the type of expression system (i.e., the type of cells) that are used and can be selected by one skilled in the art, given the benefit of the present disclosure.

In certain embodiments, approaches provided by this disclosure include making cells that express a recombinant HRG transporter by introducing into them an expression vector encoding the HRG transporter. For ease of reference, cells engineered to express a heterologous HRG according to the method of the invention are from time to time referred to herein as “rHRG+” cells.

In one embodiment, the method comprises providing a plurality of distinct samples comprising rHRG+ cells. In one embodiment, each sample expresses the same HRG. In alternative embodiments, some or all of the samples express a different HRG. The plurality of rHRG+ samples is configured so as to be amenable for high throughput screening (HTS). In certain embodiments, the samples are divided into a plurality of reaction chambers, such as wells in a plate. Any multi-well plate or other container can be used. In certain approaches, one or more 384-wells plates are used.

The method includes exposing each rHRG+ sample to a heme analog that, when transported into the cell or otherwise affecting the HRG function results in a detectable change, such as a detectable phenotype and/or signal. In embodiments, the heme analog has a detectable component, or the heme analog is toxic, thus resulting in an altered growth phenotype. In one embodiment, the toxic heme analog is lethal to the cells. Changes in cell viability subsequent to exposure to the heme analog can be measured using any suitable approach, including but not limited to determining a change, or a lack of a change, in optical density. In various embodiments, labeled heme and/or labeled heme analogs, and/or labeled test agents can be used. In one embodiment, the label is a radioactive label.

In embodiments, the effect of a test agent on a recombinant, heterologous heme transporter can be compared to a reference. Any suitable control can be used as a reference, including but not limited to a cell culture to which a test agent has not been added, or to which an agent with a known effect on the heme transporter is added, or the reference can be a standardized reference, such as a known value for, for example, optical density, or any other parameter that relates to cell viability and/or a detectable signal. In embodiments, the reference is a positive control, or a negative control.

In one embodiment, the toxic heme analog is one in which a toxic compound has been exchanged for iron in the heme complex. In one embodiment, the toxic compound is a metal agent. In embodiments, the metal gallium; thus the invention includes use of agents such as Gallium protoporphyrin IX (GaPPIX) as a toxic heme analog. Non-toxic heme analogs, such as zinc mesoporphyrin (ZnMP) can also be used, depending upon the nature of the organisms used in the assays of the invention and the method of detection of a change in HRG function.

In various aspects, methods are provided which comprise providing a plurality of rHRG+cell samples, and mixing each of the samples cells concurrently or sequentially with i) the heme analog; and ii) a test agent. The method is thus designed to determine whether or not the test agent can affect the function of the HRG. In one embodiment, the test agent inhibits or prevents appearance of a detectable change, such as a change in cell viability. For instance, if after exposure to the heme analog and the test agent there is no change in cell viability as compared to a suitable control (i.e., cells exposed only to the heme analog or the test agent), it can be concluded that the test agent is not an antagonist of the HRG in the sample. If instead, for example, the cell viability increases as compared to a control, it can be concluded that the test agent competed with the heme analog, or otherwise blocked the HRG from transporting the heme analog into the cell, the latter of which can be caused by allosteric effects. Thus, such a test agent is a candidate for use in any of a variety of applications, including but not necessarily limited to use as antagonists of an HRG, such as HRG-4, which is the prototypical C. elegans heme transporter. Further, because parasitic nematodes and the kinetoplastids acquire heme from the environment, it is anticipated that the test agents identified by the invention will be useful for such non-limiting embodiments as targeting the heme transport pathway for the treatment of any helminth infections, Trypanosomiasis, intestinal nematodes, kinetoplastid diseases, lymphatic filariasis, onchocerciasis, and Leishmaniasis, as well as human genetic disorders of heme and iron metabolism. Any HRG transporter expressed by any of organisms that cause the foregoing infections can be expressed heterologously using the compositions and methods of the invention. In embodiments, the HRG transporter is a C. elegans HRG transporter, such as C. elegans HRG-1 or HRG-4. Other HRG transporters that can be used will be apparent to those skilled in the art, given the benefit of the present disclosure.

It will be recognized from the foregoing that test agents that do not affect the activity of the heme analog on the rHRG+ samples are, in certain embodiments, not considered to be candidates for the aforementioned uses, and thus can be eliminated as candidates, or considered for other purposes.

It is plausible that the test agent will function to accelerate transport of the heme analog into the rHRG+ cells in the samples. In such a case, the test agent can be considered as a candidate therapeutic agent for use in treating conditions that relate to inefficient heme transport. The function/mechanism of such agents may be unknown. For example, they may function as HRG agonists by way of allosteric interactions. In particular, they may bind to a location of the HRG that is different from its heme binding location, or they may be directed to a non-HRG target, thus affecting heme intake in any manner that is measurable in the assays that are a subject of this invention.

It will also be apparent from the foregoing that the present invention provides novel compositions. The novel compositions include but are not limited to a plurality of rHRG+ cell samples divided into separate reaction containers, wherein the reaction containers can further comprise a heme analog, and test agents.

The following specific examples are provided to illustrate the invention, but are not intended to be limiting in any way.

### EXAMPLE 1

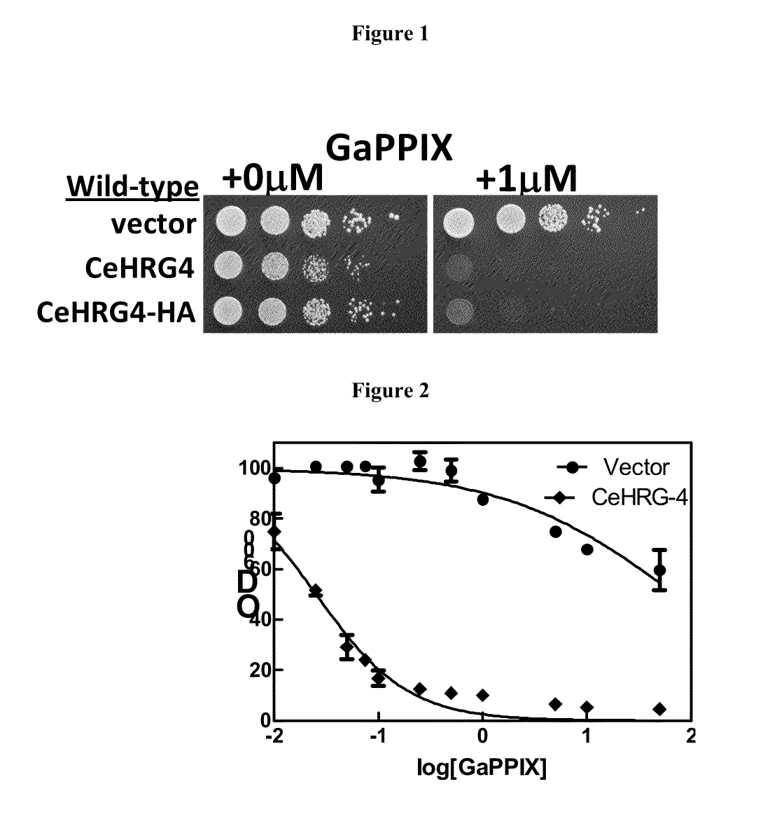

This Example provides a demonstration of a simple, powerful growth assay to identify small molecule antagonists of heme transporters. In developing a HTS-compatible cell-based assay to identify test agents that can affect the function of heme transporters we used a yeast growth assay which exploited the ability of ectopically expressed heme transporters to transport the toxic heme analog GaPPIX into wild-type yeast. Because gallium cannot undergo oxidation-reduction reactions like iron, mis-incorporation of GaPPIX into hemoproteins is cytotoxic. As shown in FIG. 1, wild-type yeast expressing either HA tagged or untagged C. elegans HRG-4 are extremely sensitive to GaPPIX. Dose-response growth curves demonstrate that HRG-4 expressing yeast have >2,000-fold lower relative IC₅₀ (half maximal inhibitory concentration) value for GaPPIX than yeast transformed with the vector alone (FIG. 2). As we have demonstrated in C. elegans, GaPPIX toxicity in HRG-4 expressing yeast can be alleviated by addition of excess heme which effectively competes with GaPPIX for uptake (not shown).

Thus, data presented in this Example demonstrate an embodiment of the method. We have successfully used this system to screen libraries of test agents and have identified numerous candidates for use as HRG antagonists.

While the invention has been described through specific embodiments, routine modifications will be apparent to those skilled in the art and such modifications are intended to be within the scope of the present invention.

We claim:

1. A cell culture comprising a plurality of cells which express a recombinant heterologous eukaryotic heme transporter, wherein the plurality of cells are divided into a plurality of reaction chambers, wherein each reaction chamber in the plurality of reaction chambers comprises a distinct test agent, wherein the test agent is a candidate for use as a modulator of the eukaryotic heme transporter, and wherein each reaction chamber in the plurality of reaction chambers further comprises a toxic heme analog.

2. The cell culture of claim 1, wherein the recombinant heterologous eukaryotic heme transporter is a eukaryotic heme transporter that is endogenously expressed by a human or non-human animal parasite.

3. The cell culture of claim 2, wherein the plurality of reaction chambers comprises at least 384 reaction chambers, and wherein each reaction chamber contains a distinct test agent.

4. A method of identifying a modulator of a eukaryotic heme transporter, the method comprising providing a culture of cells separated into a plurality of reaction chambers, wherein the cells in the culture express a recombinant heterologous eukaryotic heme transporter, adding a toxic heme analog and at least one distinct test agent to each of the reaction chambers, and incubating the cells with the toxic heme analog and the distinct test agent for a period of time, wherein a change in toxic effect of the toxic heme analog relative to a reference is indicative that the distinct test agent is a modulator of the eukaryotic heme transporter.

5. The method of claim 4, wherein the change in the toxic effect of the toxic heme analog comprises reduced lethality of the toxic heme analog, thereby identifying the test agent as an antagonist of the eukaryotic heme transporter.

6. The method of claim 4, wherein the plurality of reaction chambers comprises at least 384 reaction chambers.

7. The method of claim 4, wherein the recombinant heterologous eukaryotic heme transporter is a eukaryotic heme transporter that is endogenously expressed by a human or non-human animal parasite.
